(19) United States
(12) Patent Application Publication
(10) Pub. No.: US 2008/0119588 A1
(43) Pub. Date: May 22, 2008

(54) Novel desensitizing and fluoridizing dental varnishes

(76) Inventors: Jan A. Orlowski, Altadena, CA (US); David V. Butler, West Covina, CA (US); Lindsay K. McKinley, La Mirada, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/603,952

(22) Filed: Nov. 22, 2006

(51) Int. Cl. A61K 6/083

(52) U.S. Cl. 523/115

(57) ABSTRACT

Disclosed are dental varnishes comprising hydrogenated wood rosin and/or gum rosin dissolved in C₂-C₄ alcohols, C₅-C₇ hydrocarbons, or mixtures thereof, for use in preventing tooth decay and/or alleviating tooth sensitivity. Such varnishes may, optionally, incorporate therapeutic, antimicrobial and sensory enhancing ingredients.

### BACKGROUND OF THE INVENTION

1. Field of the Invention

This invention relates to dental varnishes, more specifically desensitizing and teeth fluoridizing varnishes comprising a solution of hydrogenated rosins in a volatile solvent.

2. Description of the Related Art

The oldest type of dental varnishes comprised solutions of copal-type rosin, a fossilized plant-derived material, in ethyl ether, chlorinated hydrocarbons and, in later versions, in alcohols. These varnishes were almost exclusively used as coatings over the exposed dentin prior to placement of amalgam- or phosphate-type restoratives. They were not suitable for use under the novel resin based restoratives because copal acts as an inhibitor of the polymerization process, negatively influencing the cure of the restorative material and, in consequence, the quality of the restoration. Incomplete cure of the material could also cause postoperative discomfort and serious damage to the pulp. Copal varnishes, although generally beneficial in preventing marginal leakage of the new restorations, did not offer benefits of tooth fluoridation and, therefore, did not provide a desirable, additional measure of protection against secondary decays.

Copal-type resin based varnishes were followed by varnishes based on synthetic, primarily polyamide-type polymers. Examples of such varnishes are described in U.S. Pat. No. 4,396,378. Synthetic varnishes did not inhibit the polymerization process of resin based restorative materials. Therefore, such varnishes were suitable for use under all known types of restoratives. Because of their low specific gravity and low viscosity, they were unable to maintain fluoride salts in suspension. Their role was, therefore, limited to sealing dentin tubulae without providing the benefits of fluoridizing potentially vulnerable tooth surfaces.

Newer types of dental varnishes encompass formulations comprising, in addition to film formers (natural or synthetic resin), therapeutic agents, to effect desirable changes in the chemistry of teeth, reduce tooth sensitivity and to prevent tissue irritation or damage, resulting from contact with dental materials.

The most common among therapeutic additives in modern dental varnishes are fluoride salts, particularly sodium fluoride. The most common fluoride containing dental varnishes are those comprising colophony-type rosins as a film former, ethyl alcohol as a medium/solvent and sodium fluoride as the main, or only, therapeutic agent. Colophony is a natural product derived from trees and, as such, its characteristics frequently vary from one lot to another. The differences often include properties relevant for the performance or esthetics of the varnish and also may affect its potential to irritate oral tissues. A desirable consistency of the varnish is important to achieve proper film thickness of the coating, to facilitate handling and, most of all, to prevent sedimentation of fluoride salts and other dispersed components of the formulation. Known disadvantages of these varnishes include their slow solvent release, resulting in a slow cure, unsightly appearance, and their unpleasant taste.

### SUMMARY OF THE INVENTION

In accordance with one embodiment, there is provided a dental varnish for preventing or alleviating tooth sensitivity and reducing incidences of decay, wherein the varnish comprises a film-forming resin and a solvent, where the solvent preferably comprises one or more C₂-C₄ aliphatic alcohol or one or more C₅-C₇ hydrocarbon, or mixtures thereof. The film forming resin comprises hydrogenated rosin, partially polymerized hydrogenated rosin, neutralized or partially neutralized hydrogenated rosin, or combinations thereof. In one embodiment, the solvent comprises an azeotropic or near azeotropic mixture of solvents, preferably, ethyl alcohol and n-hexane.

### DETAILED DESCRIPTION OF THE PREFERRED EMBODIMENT

Disclosed herein is a new type of dental varnish, which while preserving advantages and functions of prior art varnishes, preferably further enhances their relevant characteristics, particularly esthetics, taste, and biocompatibility (reduction or absence of oral tissue irritation).

Rosin, a plant-derived product, comprises organic acids whose carboxylic groups may cause oral tissue irritation. Also, it has been found that unmodified rosins, such as colophony-type, may cause skin sensitization in rare cases. Among other negative aspects of using unmodified rosins, particularly in dental applications, are their unpleasant taste, unsightly color, low and often inconsistent softening point, presence of insoluble impurities and slow solvent release. These shortcomings contribute to inferior, or compromised, final product performance and user acceptance.

The dental varnishes disclosed herein use hydrogenated, unpolymerized or polymerized, rosin. Neutralization or partial neutralization of rosin is optional but desirable for certain types of rosins.

Hydrogenated rosins and their polymers, including those partially or fully neutralized, are available free of visible impurities, in uniform and light colored commercial forms, allowing for formulating varnishes which are virtually invisible on teeth after cure. Hydrogenated rosins employed in varnishes according to preferred embodiments are those in which 50%-100% of the originally present unsaturated bonds have been eliminated by hydrogenation, including about 50%-74% (also referred to as partially hydrogenated) and 75%-100% (also referred to as highly hydrogenated). Preferred varnishes comprise at least about 20% rosin by weight, including about 30%, about 35%, about 40%, about 45%, about 50%, about 55%, about 60%, about 65%, about 70%, and about 75%, and also including percentages between these recited percentages, as well as ranges bordered on each end by recited percentages, such as about 30-70%. In one embodiment, the resin mixtures includes one or more other natural or artificial resins, including but not limited to, colophony, cumarone, copal, and polyamide resins. Such other resins are preferably present at about 1-20%, including about 5%, about 10% and about 15%, including percentages between these recited percentages, as well as ranges bordered on each end by recited percentages. Preferred polyamides include those which are a reaction product of diamines, including aliphatic diamines, with fatty acids.

In certain preferred embodiments, concentration of such film formers in varnishes featuring optimal or near optimal consistency is very high, in a range of 50-75% by weight which contributes to their expedient cure, further enhanced by fast solvent release. Good mechanical characteristics of the varnish allow extended fluoride release time, when compared to conventional colophony-type formulations, therefore, improving their efficacy as teeth fluoridizing and desensitizing agents.

In certain preferred embodiments, the solvent component of a varnish comprises one or more alcohols and/or one or more hydrocarbons. In some embodiments, the solvent comprises a mixture of alcohol and hydrocarbons at azeotropic or near azeotropic proportions, including, but not limited to: a mixture of ethyl alcohol (21% by weight) with n-hexane (79% by weight), having boiling point of 58.7° C. at 760 mm Hg; a mixture of isopropyl alcohol (23% by weight) with n-hexane (77% by weight), having boiling point of 62.7° C. at 760 mm Hg.; and pure ethyl alcohol, having boiling point of 78.3° C. at 760 mm Hg. Solutions of hydrogenated rosin in such mixtures provide fast curing, and less- or non-irritating varnishes. The relative weights given above relate only to azeotropic mixtures, and are not intended to restrict the absolute or relative amounts of alcohol and/or hydrocarbon in the varnish formulations. Varnish formulations may include individual solvents (e.g. ethyl alcohol only), or mixtures of alcohols, individual hydrocarbons or their mixtures, or mixtures of alcohols with hydrocarbons.

Preferred alcohols include C₂-C₄ alcohols, including C₃ alcohols, wherein said alcohols may be linear, branched and/or cyclic. Preferred alcohols include ethyl alcohol, propyl alcohol (including its isomers n-propyl alcohol and isopropyl alcohol), butyl alcohol (including its isomers, namely n-butyl alcohol, sec-butyl alcohol, iso-butyl alcohol, and t-butyl alcohol), and blends thereof. Use of alcohols outside the C₂-C₄ range is also contemplated. Preferred hydrocarbons include C₅-C₇ hydrocarbons, including C₆ hydrocarbon compounds, wherein said hydrocarbons may be linear, branched and/or cyclic, and may be alkanes and/or alkenes. A hydrocarbon component may comprise a single hydrocarbon or a blend of two or more hydrocarbons. Specific preferred hydrocarbons include isopentane. n-pentane, n-hexane, isohexanes, cyclohexene, cyclohexane, methylcyclopentane, n-heptane, methyl cyclohexane, 2,5-dimethylhexane, cyclohexene, methyl cyclohexene, 1-heptene, and mixtures thereof. Use of hydrocarbons outside the C₅-C₇ range is also contemplated.

In a preferred embodiment, the varnish comprises at least about 15% solvent by weight, including about 18%, about 19%, about 20%, about 21%, about 22%, about 23%, about 24%, about 25%, about 30%, and about 35%, and also including percentages between these recited percentages, as well as ranges bordered on each end by recited percentages. In a preferred embodiment, the varnish comprises at least about 4% alcohol component by weight, including about 5%, about 6% about 7%, about 8%, about 9%, about 10%, about 11%, about 12%, about 13%, and about 14%, also including percentages between these recited percentages, as well as ranges bordered on each end by recited percentages. In a preferred embodiment, the varnish comprises at least about 10% hydrocarbon component by weight, including about 11%, about 12% about 13%, about 14%, about 15%, about 16%, about 17%, about 18%, about 19% and about 20%, also including percentages between these recited percentages, as well as ranges bordered on each end by recited percentages. Certain embodiments of the invention may include components present at concentrations above and below the preferred concentrations recited.

Preferred fluoridizing agents include sodium fluoride, stannous fluoride, sodium monofluorophosphate, zinc hexafluorosilicate, and sodium hexafluorosilicate. When present, there is preferably about 0.1-10% fluoridizing agent by weight, including at least about 1%, about 2%, about 2.5%, about 3%, about 3.5% about 4%, about 4.5%, about 5%, about 5.5%, and about 6%, also including percentages between these recited concentrations, as well as ranges bordered on each end by recited percentages, such as about 2.5%-6% and about 3-5%.

The varnish formulations optionally comprise one or more of the following: sweeteners, such as xylitol, sorbitol, aspartame, sodium saccharin, and mixtures thereof; flavorings, including but not limited to, peppermint, watermelon, wintergreen, spearmint, cherry, citric acid, orange, strawberry, vanilla, coconut, bubble gum flavors and mixtures thereof; coloring agents; and organic and/or inorganic fillers or thickeners. When a sweetener is present, its concentration is preferably about 0.5-3% by weight, including about 1%, 1.5%, 2%, and 2.5% by weight, or other quantity sufficient to impart an improved palatability to the varnish. When flavorings are present, they are preferably present at about 1-4% by weight, including about 1.5-3%, including about 2% and about 2.5% and values therebetween. When a filler or thickener is present, such as silica, it is preferably present at about 2-10%, including about 3-6% by weight.

The varnishes described herein may be applied to teeth using a suitable applicator, preferably a brush, as is well understood by those skilled in the art. Methods of using the varnish to seal a tooth include applying the varnish to a tooth and allowing the solvent to evaporate, leaving a film, which adheres to the treated surfaces.

The invention is further described in the examples, which are given solely for the purpose of illustration, and are not intended to limit its scope as defined in the patent claims. Unless stated otherwise, all percentages given below, and elsewhere herein, are by weight.

### EXAMPLES

### Example 1

The varnish formulation consisted of:

| | |
|---|---|
| Highly hydrogenated wood rosin | 29.8% |
| N-Hexane | 23.5% |
| Silica | 19.3% |
| Polyamide resin (a condensation product of | 11.3% |
| ethylenediamine with fatty acids) | |
| Ethyl Alcohol | 6.3% |
| Sodium Fluoride | 4.3% |
| Watermelon Flavor | 3.0% |
| Xylitol | 2.2% |
| Sodium Saccharine | 0.3% |

The formulation has shown an adequate consistency to maintain sodium fluoride and xylitol in suspension. After forcing sedimentation by centrifuging or prolonged storage at elevated temperatures, a uniform suspension was easily restored by gentle stirring. Curing time at 37° C. was approximately six minutes. Color and translucency were judged excellent.

### Example 2

The varnish formulation consisted of:

| | |
|---|---|
| Partially neutralized, highly hydrogenated wood rosin | 70.4% |
| Hexanes (a mixture of isomers) | 11.8% |
| Sodium Fluoride | 4.9% |
| Silica | 3.7% |
| Cherry Flavor | 3.1% |
| Ethyl Alcohol | 3.1% |
| Xylitol | 2.7% |
| Sodium Saccharine | 0.3% |

The formulation has shown an adequate consistency to maintain sodium fluoride and xylitol in suspension. After forcing sedimentation by centrifuging or prolonged storage at elevated temperatures, a uniform suspension was easily restored by gentle stirring. Curing time at 37° C. was approximately five minutes. Color and translucency were judged excellent.

### Example 3

The varnish formulation consisted of:

| | |
|---|---|
| Partially neutralized, highly hydrogenated wood rosin | 67.6% |
| Ethyl Alcohol | 18.5% |
| Silica | 5.3% |
| Sodium Fluoride | 4.7% |
| Xylitol | 2.6% |
| Bubble Gum Flavor | 1.3% |

The formulation has shown an adequate consistency to maintain sodium fluoride and xylitol in suspension. After forcing sedimentation by centrifuging or prolonged storage at elevated temperatures, a uniform suspension was easily restored by gentle stirring. Curing time at 37° C. was approximately eight minutes. Color and translucency were judged excellent.

### Example 4

The varnish formulation consisted of:

| | |
|---|---|
| Highly hydrogenated wood rosin | 34.4% |
| N-Hexane | 26.5% |
| Silica | 22.4% |
| Isopropyl Alcohol | 7.9% |
| Sodium Fluoride | 4.5% |
| Xylitol | 2.5% |
| Bubble Gum Flavor | 1.3% |
| Sodium Saccharine | 0.50% |

The formulation has shown an adequate consistency to maintain sodium fluoride and xylitol in suspension. After forcing sedimentation by centrifuging or prolonged storage at elevated temperatures, a uniform suspension was easily restored by gentle stirring. Curing time at 37° C. was approximately nine minutes. Color and translucency were judged excellent.

### Example 5

The varnish formulation consisted of:

| | |
|---|---|
| Silica | 31.3% |
| Partially hydrogenated gum rosin | 27.7% |
| Hexane | 23.55% |
| Ethyl Alcohol | 8.5% |
| Sodium Fluoride | 4.0% |
| Cherry Flavor | 2.54% |
| Xylitol | 2.2% |
| Sodium Saccharine | 0.21% |

The formulation has shown an adequate consistency to maintain sodium fluoride and xylitol in suspension. After forcing sedimentation by centrifuging or prolonged storage at elevated temperatures, a uniform suspension was easily restored by gentle stirring. Curing time at 37° C. was approximately five minutes. Color and translucency were judged excellent.

What is claimed is:

1. Dental varnish for preventing or alleviating tooth sensitivity and/or reducing or preventing occurrences of tooth decay comprising: a hydrogenated wood or gum rosin, and a solvent comprising of at least one C₂-C₄ aliphatic alcohol or their mixtures with at least one C₅-C₇ hydrocarbon.

2. The varnish of claim 1, wherein said rosin is hydrogenated to a varied degree.

3. The varnish of claim 1, wherein said hydrogenated rosin is partially polymerized.

4. The varnish of claim 1, wherein such hydrogenated rosin is partially or completely neutralized.

5. The varnish of claim 1, wherein said solvent comprises an azeotropic or near azeotropic mixture of alcohols with C₅-C₇ hydrocarbons.

6. The varnish of claim 1, wherein said solvent comprises an azeotropic or near azeotropic mixture of ethyl or isopropyl alcohol with n-hexane, its isomers or mixtures thereof.

7. The varnish of claim 1, wherein said solvent comprises ethyl alcohol or isopropyl alcohol or a mixture thereof.

8. The varnish of claim 1 containing 0.1%-10% by weight of fluoride salt selected from the group consisting of sodium fluoride, potassium fluoride, sodium monofluorophosphate, stannous fluoride, zinc hexafluorosilicate, sodium hexafluorosilicate and potassium hexafluorosilicate.

9. The varnish of claim 1, wherein said rosin component is present at a concentration of 25%-85% by weight.

10. The varnish of claim 1, wherein said solvent has a boiling point at atmospheric pressure below 100° C.

11. The varnish of claim 1 additionally containing coloring or opaquing agents.

12. The varnish of claim 1 additionally containing sensory enhancing agents, such as sweeteners and flavors.

13. The varnish of claim 1 additionally containing inorganic and/or organic fillers.

14. The varnish of claim 13, wherein said inorganic filler is silica.

15. The varnish of claim 1 further comprising 1%-25% by weight of other type natural or synthetic resin.

16. The varnish of claim 15, wherein said natural resin is a rosin of colophony-, copal- and/or cumarone-type.

17. The varnish of claim 15, wherein said synthetic resin comprises a polyamide.

18. The varnish of claim 17, wherein said polyamide is a reaction product of aliphatic diamines with fatty acids.

19. The varnish of claim 1, wherein the rosin is 50%-100% hydrogenated.

20. The varnish of claim 1, wherein the rosin is 75%-100% hydrogenated.
